(19) United States
(12) Patent Application Publication
(10) Pub. No.: US 2011/0044919 A1
(43) Pub. Date: Feb. 24, 2011

(54) Compositions With Perfluorinated Ingredients

(76) Inventors: Paolo U. Giacomoni, Commack, NY (US); Phillip Cummins, Livingston, NJ (US); Peter J. Lentini, West Babylon, NY (US); Manuel L. Tan, Westbury, NY (US); John R. Castro, Huntington Station, NY (US); Ismail Ahmed Syed, Ronkonkoma, NY (US); Garrett William Vanderover, Bellerose, NY (US)

Correspondence Address: THE ESTEE LAUDER COS, INC, 155 PINELAWN ROAD, STE 345 S, MELVILLE, NY, 11747, US

(21) Appl. No.: 12/671,709

(22) PCT Filed: Aug. 11, 2008
(86) PCT No.: PCT/US08/72751

§ 371 Date: Nov. 11, 2010

Related U.S. Application Data
(60) Provisional application No. 60/955,883, filed on Aug. 15, 2007.

(51) Int. Cl. A61K 8/72; A61K 8/70; A61K 8/58; A61K 8/30; A61Q 17/04; A61Q 19/00

(52) U.S. Cl. 424/60; 514/759; 514/672; 514/578; 424/78.02; 424/59

(57) ABSTRACT

The invention is directed to a cosmetic composition comprising at least one perfluorinated component and at least one aromatic component, wherein the aromatic component is present in an amount sufficient to compatibilize the fluorinated component in the cosmetic composition; transfer resistant, long wearing or other types of cosmetic or pharmaceutical compositions containing the components and a method for compatibilizing a fluorinated component in a cosmetic composition using an aromatic component, and cosmetic compositions containing at least one perfluorinated amide.

This application is a national stage filing of PCT/US2008/072751, filed Aug. 11, 2008, and claims priority from US60/955,883, filed Aug. 15, 2007.

### TECHNICAL FIELD

The invention is in the field of cosmetic or pharmaceutical compositions in anhydrous or emulsion form containing a perfluorinated component and an aromatic component.

### BACKGROUND

In the last ten to twenty years, the use of polymers and other new synthetic ingredients in cosmetics has facilitated advances in cosmetic properties such as the length of time a cosmetic applied to skin will remain on the skin (a property often referred to as “wear”) and the ability of the composition to adhere to the keratinous substrate to which it is applied. Today's women most often work outside the home and otherwise lead very busy lives compared to women a century ago. A large majority of these women use cosmetics. Most of them want cosmetics that wear well because they don't have time to constantly check themselves in the mirror to make sure their lipstick is still fresh looking, or reapply cosmetics many times throughout the day. The so-called transfer resistant lipsticks were first commercialized in the mid-90s. The term “transfer resistant” referred to lipsticks that did not transfer from the lips to cups, eating utensils, fabrics, or other skin when in contact with the lips. These transfer resistant lipsticks wore so well that they often remained on the lips throughout the day without the need for reapplication. However, one problem with the early transfer resistant lipsticks is that they were very dry on the lips and provided only a matte finish. Technology was such that it was not possible to provide a transfer resistant lipstick that contained any appreciable moisturizing ingredients such as oils because the transfer resistant finish would be compromised. In addition, the extreme matte finish of these products was not desirable to many lipstick users. Many of them wanted the lipstick finish on the lips to provide a dewy, somewhat glossy finish that resembles the look of youthful skin. The transfer resistant lipsticks addressed the need gap provided by lipsticks that wore off too easily but created new need gaps—the desire for a moisturizing lipstick that was not so matte looking on the lips.

The cosmetics industry continued in its efforts to develop gold standard transfer resistant lipsticks—formulas that provided optimal wear, were as comfortable on the lips as a standard non-transfer lipstick, and which also provided a glossy finish. The desired result has proven to be elusive. The cosmetics industry attempted to fill this need gap with two-pack products, that is, products that were applied to the lips in two separate steps. In the typical first step a color coat containing the pigments and film formers was applied to lips and allowed to dry. In the second step a glossy topcoat that would not react or solvate the previously applied color coat was applied on top and provided the shiny finish. The two pack products have been successful despite their obvious drawbacks. Cosmetics companies are still on a quest for a transfer resistant or long wearing color cosmetics such as lipstick, which provides a shiny finish, is moisturizing on the lips, all in one formula.

Color cosmetics formulators know that there are certain cosmetic raw materials that provide shine when formulated into cosmetic products. Silicones substituted with aromatic rings and fluorinated materials are two examples of ingredients that are known to provide glossy properties when used in cosmetics. Fluorinated hydrocarbons, fluorinated silicones, or fluorinated esters have been taught for use in preparing long wearing or transfer resistant color cosmetics. Fluorinated materials are particularly effective in providing shine but are very incompatible with almost all ingredients used in cosmetic formulas due to their extreme hydrophobicity and/or lipophobicity. This incompatibility is why such fluorinated materials generally cannot be used in cosmetic formulas in amounts sufficient to provide appreciable long lasting shine. Nor do such currently available materials provide optimum adhesion on keratinous surfaces such as skin, lips, lashes, or hair. In some cases they are used to provide a temporary gloss on the surface of a lip color due to phase separation. That is, due to its incompatibility in the formula it separates from the remaining ingredients when the lipstick is applied to the lips. This phase separation causes the fluorinated material to migrate to the surface of the applied composition and provide a glossy finish that fades fairly quickly.

It has been discovered that certain types of fluorinated components that, alone, are incompatible with most cosmetic ingredients can be solvated or compatibilized with cosmetic ingredients if a second aromatic component, specifically a compound, oligomer, or polymer containing homonuclear or heteronuclear aromatic substituted groups is present.

It is an object of the invention to provide cosmetic compositions that are transfer resistant or long wearing, and in the case of lipsticks, shiny, and comfortable on the lips.

It is a further object of the invention to provide compatible cosmetic compositions that contain at least one perfluorinated component and at least one aromatic component wherein the aromatic component compatibilizes the perfluorinated component in the cosmetic composition such that a commercially acceptable, stable, compatible product can be formulated.

It is a further object of the invention to provide a method for enabling the use of perfluorinated components in cosmetic compositions when they would otherwise be incompatible by compatibilizing the perfluorinated components with an aromatic component, more specifically, an aromatic component that contains at least one substituted homonuclear or heteronuclear aromatic ring.

It is a further object of the invention to provide a method for compatibilizing fluorinated compounds in the form of a fluorinated head and a hydrophilic hydrocarbon tail, or in the form of a fluorinated head and a lipophilic hydrocarbon tail with cosmetic ingredients by using aromatic components.

It is a further object of the invention to provide a topcoat for a color cosmetic composition comprising at least one fluorinated component and at least one aromatic component, and a two pack color cosmetic composition comprising a color coat and a topcoat containing the at least one fluorinated component and at least one aromatic component.

A further object of the invention is to provide a carrier composition for pharmaceutical compounds containing fluorinated components and aromatic components.

### SUMMARY OF THE INVENTION

The invention is directed to a cosmetic composition comprising at least one perfluorinated component which may be a compound, oligomer, or polymer. The perfluorinated component alone is incompatible with one or more of the other cosmetic ingredients present in the formula. The composition also contains at least one aromatic component that may be a compound, oligomer, or polymer, wherein the aromatic component is present in an amount sufficient to compatibilize the perfluorinated ingredient with the remaining ingredients in the composition.

The invention is also directed to a long wearing or transfer resistant cosmetic composition comprising at least one perfluorinated component and at least one aromatic component present in an amount sufficient to compatibilize the perfluorinated component with the other ingredients in the cosmetic composition.

The invention is further directed to a transfer resistant or long wearing cosmetic composition comprising at least one perfluorinated component, at least one aromatic component, at least one volatile solvent, at least one nonvolatile oil, and particulates.

The invention is further directed to a method for compatibilizing a fluorinated component in a cosmetic composition comprising using an aromatic component to compatibilize the fluorinated component in the composition. In one preferred embodiment the fluorinated component is dispersed in the aromatic component prior to formulating into a cosmetic composition.

The invention is further directed to a cosmetic composition comprising at least one fluorinated amide.

### DETAILED DESCRIPTION

All percentages used herein are percentages by weight unless otherwise indicated. The compositions of the invention may be in anhydrous or emulsion form, or in the form of aqueous non-aqueous solutions or dispersions. They may be in the form of liquids, solids, or semi-solids at room temperature (25° C.). If water is present, the compositions may contain from about 0.1 to 99%, preferably from about 0.5 to 95%, more preferably from about 1 to 90% by weight of the total composition.

### I. The Perfluorinated Component

The composition of the invention contains at least one perfluorinated component that may be in the form of a compound, oligomer, polymer, or combination thereof. The perfluorinated ingredient may be present in the form of a liquid, semi-solid, or solid, and in amounts ranging from about 0.1 to 90%, preferably from about 1 to 85%, more preferably from about 2 to 80% by weight of the total composition. The perfluorinated component alone may be generally incompatible with one or more of the ingredients in the cosmetic formulation. The term “incompatible” means that the ingredient causes an internally incompatible formula that is not commercially acceptable. An internally incompatible formula is often referred to by seasoned cosmetic formulators as a formula that “falls apart”. This incompatibility or instability may be due to the chemical nature of the actual perfluorinated ingredient itself or due to the amount in which the ingredient is present in the composition. For example, in order to achieve the desired properties of gloss or adhesion it may be necessary include the perfluorinated component in the composition in more than a token amount, and it is that amount that is incompatible with one or more of the remaining cosmetic ingredients. Or in another case due to its chemical composition the perfluorinated component itself is incompatible in the formula in any amount, even very small amounts like trace amounts. In either case, the perfluorinated component is such that if used in the cosmetic formulation to achieve the desired properties for which it was included it would not provide a commercially acceptable product. Suitable perfluorinated compounds, oligomers, or polymers or mixtures thereof are further described herein.

A. Perfluorinated Compound

The perfluorinated component may be in the form of a compound. In such a case, the perfluorinated compound is the reaction product of various fluorinated or non-fluorinated radicals such as perfluoroalkyl groups, alkyl groups, alkylene groups, alkoxy groups, amide groups, and the like provided that the compound contains at least one fluorine substitution. More particularly, the perfluorinated compound may contain a combination of radicals, further defined below as Radical I, II, III; provided that the compound contains at least one Radical I. Radical I may also be referred to as the “fluorinated head” of the compound, and Radicals II and III the tail of the compound.

The perfluoroalkyl group may have from about 2 to 50,000 carbon atoms, preferably from about 2 to 100, more preferably from about 2 to 50 carbon atoms. All of the hydrogen atoms may be substituted with fluorine atoms (often referred to as fluorocarbons), or any portion of the hydrogen atoms present may be substituted with fluorine atoms, say fluorine substitutions on the perfluoroalkyl chain ranging from 1 to 99%. In the latter case, radicals having both hydrogen and fluorine substitutions are referred to as fluorohydrocarbons. In addition, the perfluoroalkyl group may be substituted with other substituents such as hydroxyl, alkyl, hydroxyalkyl, alkoxy, non-fluorine halogens, alkyl ethers, and the like. For example, the perfluoroalkyl radical may have the general formula (Radical I);

CXYZ—(CXY)ₐ— Radical I.

Wherein the index, “a” ranges from 1 to 50,000, preferably from 1 to 500, more preferably from 1 to 100, most preferably where “a” is 1 to 30; and each X, Y, or Z is independently hydrogen, fluorine, C₁₋₁₀ alkoxy, C₁₋₁₀ alkyl, hydroxyl, halogens other than fluorine, and the like, with the proviso that Radical I contains at least one substituted fluorine atom; more specifically where the radical contains a number of substituted fluorine atoms sufficient to confer incompatibility when the perfluorinated compound, alone, is incorporated into a cosmetic composition. In one preferred embodiment, X, Y, and Z are all fluorine. In one preferred embodiment of Radical I, index “a” is such that the radical is a so-called perfluorinated “fatty” radical, having from about 6 to 22 carbon atoms, such as perfluorohexyl, perfluoroheptyl, perfluorooctyl, perfluorononyl, perfluorodeca, perfluorododeca, perfluorococoyl, perfluorocetyl, perfluoroisocetyl, perfluorocetyl, perfluoroisostearyl, perfluorostearyl, or perfluorobehenyl. Preferably, Radical I is a fully fluorinated fatty perfluoroalkyl radical.

Examples of perfluorinated compounds comprised of Radical I include but are not limited to linear or branched perfluoroalkanes such as perfluoroethane, perfluoropropane, perfluorobutane, perfluoroisobutane, perfluoropentane, perfluorohexane, perfluoorooctane, perfluorononane, perfluorodecane, perfluorododecane, perfluoroisododecane, and/or perfluorinated alcohols such as C9-13 fluoroalcohols, C6-12 perfluoroalkyl alcohol (ethanol, propanol), and so on. In such cases the perfluorinated compound may be volatile, e.g. may have a vapor pressure of greater than about 2 mm. of mercury at 25° C. Further examples of perfluorinated compounds comprised of Radical I include further substitutions on the perfluorinated radical, such as hydroxyl, alkyl ether, alkoxy, alkoxyalkyl, alkylalkoxyalkyl, halogens other than fluorine, wherein the alkyl or alkoxy is a C₁₋₁₀ straight or branched chain alkyl.

If desired, Radical I may be combined with Radical II to form the fluorinated compound. Radical II may be an ester group, amide group, ether group, carboxyl group, urethane group, vinyl, carboxylic acid, and so on. Radical II may be selected from:

—C(O)—NR—; —C(O)—; —O—; —O—C(O)—; —C(O)—O—; OH Radical II:

where R is hydrogen, or straight or branched chain C₁₋₁₀ alkyl. In one preferred embodiment, Radical II is an amide or ester, more preferably, amide —C(O)—NR where R is hydrogen.

The perfluorinated compound of the invention may also comprise another radical, Radical III. Preferably, Radical III comprises an alkyl, alkoxylalkyl, alkylalkoxylalkyl, hydroxylalkyl, alkoxyalkyl, hydroxyalkyl, alkylhydroxyalkyl, wherein the alkyl or alkoxy is a C₁₋₁₀ straight or branched, saturated or unsaturated alkyl, and so on. For example, Radical III may have the general formula:

—(CMN)a-CLMN Radical III:

wherein index “a” is as defined above for Radical I, and each L, M or N is independently hydrogen, hydroxyl, alkoxy, alkylalkoxyalkyl, alkylalkoxy, or alkoxyalkyl, wherein the alkyl or alkoxy is a C₁₋₁₀ straight or branched chain; or alkylene glycol having the general formula:

—[—O—(CH₂)ₓ]_{y}—

wherein x is 2, 3, 4, 5, 6, 7, 8, 9, or 10; preferably 2, 3, 4, or 5, more preferably 2, 3, or 4; and y ranges from 2 to 50,000, preferably from 2 to 400, more preferably from about 2 to 350.

Radical III may be present in straight or branched chain form, or saturated or unsaturated, and may be substituted with radicals such as halogen, including fluoro, hydroxyl, alkoxy, alkylalkoxy, alkylalkoxyalkyl, alkylene glycol, wherein the alkyl is a C₁₋₁₀ straight or branched, saturated or unsaturated alkyl.

In one preferred embodiment, the perfluorinated compound is comprised of Radicals I-II-III in combination, wherein Radical I and Radical III have essentially the same number of carbon atoms, or where the number of carbon atoms on Radicals I and III differ by no more than about 10 carbon atoms, preferably no more than about 5 carbon atoms. In another embodiment, the perfluorinated compound comprises a combination of Radicals I, II, and III wherein Radical III is a fatty alkyl or alkyl-alkylene glycol radical, specifically wherein the alkylene glycol is ethylene glycol, and the number of repeating ethylene units range from 2 to 350.

In another preferred embodiment of the invention, Radicals I and II are as set forth herein and Radical II comprises an amide, e.g. —[C(O)—NR]—; where R is hydrogen.

Examples of perfluorinated compounds that are suitable for use in the compositions of the invention include those having the following general formula:

CF₃—(CF₂)ₙ—C(O)—NH—(CH₂)ₘ—CH₃; or

CF₃—(CF₂)ₙ—C(O)—O—(CH₂)ₘ—CH₃; or

CF₃—(CF₂)ₙ—O—C(O)—(CH₂)ₘ—CH₃; or

CF₃—(CF₂)ₙ—O—(CH₂)ₘ—CH₃; or

CF₃—(CF₂)ₙ—C(O)—(CH₂)ₘ—CH₃

wherein each of m and n are independently 1 to 5,000. More preferred is wherein each n is independently 1 to 40, most preferably from 1 to 22, most preferably from 1 to 20 and wherein the n in portions corresponding to Radical I and Radical III do not vary by more than 10, preferably more than about 5.

Most preferred is a compound having the name N-octylperfluorononamide having the general formula:

CF₃—(CF₂)₇—C(O)—NH—(CH₂)₇—CH₃

Also suitable examples of perfluorinated compounds are those having the general formula:

CF₃—(CF₂)ₙ—C(O)—NH—(CH₂)ₘ—[O—(CH₂)ₓ]_{y}—Z; or

CF₃—(CF₂)ₙ—C(O)—O—(CH₂)ₘ—[O—(CH₂)_{X}]_{y}—Z; or

CF₃—(CF₂)ₙ—O—C(O)—(CH₂)ₘ—[O—(CH₂)ₓ]_{y}—Z; or

CF₃—(CF₂)ₙ—O—(CH₂)ₙ—(CH₂)ₘ—[O—(CH₂)ₓ]_{y}—Z; or

CF₃—(CF₂)ₙ—O—C(O)—[O—(CH₂)_{X}]_{y}—Z; or

CF₃—(CF₂)ₙ—C(O)—(CH₂)ₘ—[O—(CH₂)ₓ]_{y}—Z; or

wherein x is 2, 3, 4, 5, 6, 7, 8, 9, or 10, preferably 2; y ranges from 2 to 1,000, preferably from 2 to 350, more preferably from 2 to 200; and Z is hydrogen or C₁₋₁₀ alkyl, preferably methyl; and m and n are as defined above.

Further specific examples of the compounds include, but are not limited to, those set forth below:

CF₃—(CF₂)₆—C(O)—NH—CH₂—[O—CH₂CH₂]₅₅₀—CH₃; or

CF₃—(CF₂)₆—C(O)—NH—CH₂—[O—CH₂CH₂]₁₅₀—CH₃; or

CF₃—(CF₂)₆—C(O)—NH—CH₂—[O—CH₂CH₂]₂₅₀—CH₃; or

CF₃—(CF₂)₁₀—C(O)—NH—CH₂—[O—CH₂CH₂]₃₅₀—CH₃; or

CF₃—(CF₂)₁₂—C(O)—NH—CH₂—[O—CH₂CH₂]₃₅₀—CH₃; or

CF₃—(CF₂)₁₄—C(O)—NH—CH₂—[O—CH₂CH₂]₂₀₀—CH₃; or

CF₃—(CF₂)₈—C(O)—NH—CH₂—[O—CH₂CH₂]₁₀₀—CH₃; and so on.

Such perfluorinated compounds, including perfluorinated amides may be prepared as set forth in an article entitled: Surface Activity of N-Substituted Amides of Perfluoroaliphatic Monocarbonic Acids at the Interface Between Their Solutions in an Aromatic Hydrocarbon and Air, by L. A. Shits and N. A. Safronova, Institute of Physical Chemistry, Moscow, USSR; Colloidal Journal, 33 (3), pages 466-467, 1971; CA Abstracts 1971-453504, Document No. 75:53504. In particular, such fluorinated amides may be prepared by reacting equimolar quantities of perfluoropelargonic acid and primary aliphatic amines such as n-octylamine. Such perfluorinated amides may also be prepared by reacting perfluoroacyl chlorides to an excess of aqueous ammonia or an aqueous solution of amines. In particular, perfluorinated acyl chlorides are added drop-wise to an aqueous ammonia solution to provide the perfluorinated amid

e. This process is set forth in Japanese Patent No. 63077847 to Sony Corporation, published in 1986; and is also disclosed in Chem Abstracts No. 109:92293, entitled Process for the Preparation of Perfluorocarboxylic Acid Amides from Perfluoroacyl Chlorides, by Hirofumi Kondo, 1988. Another process for synthesizing such compounds is set forth in Japanese Patent No. 07112959, published as JP 1993-256991 on Jan. 14, 1993: Chem Abstracts No. 123:82851, entitled: Perfluorocarboxlic Acid Amides, Their Preparation, and Water Repellants Containing Them, by Hajime, Ito and Ueda, Kunimasa.

Perfluorinated alkyl esters that may be used in the compositions of the invention may be made by reacting a perfluorinated alcohol with a linear or branched carboxylic acid, optionally with a catalyst, in a typical esterification reaction well known in the art. For example, the carboxylic acid is heated to reflux and combined with the primary or secondary perfluoroalkanol in the presence of a catalyst (e.g. H₂SO₄) to form the ester in a reaction referred to as a Fischer esterification. Similarly, perfluorinated ethers may be made by standard etherification processes known in the art.

In the most preferred embodiment of the invention the compound is N-octylperfluorononamide, which is in the form of a solid at room temperature (25° C.)

B. Perfluorinated Oligomers or Polymers

Also suitable as the perfluorinated component are various fluorinated polymers or oligomers that may be in the form of organic polymers or silicone polymers.

1. Perfluorinated Organic Polymers

Certain types of perfluorinated organic polymers may be used in the compositions of the invention such as linear or branched perfluorinated polyethers. One type of perfluorinated polyether is a branched polymer having the general formula:

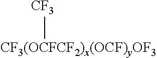

wherein x and y are each independently from about 1 to 10,000. Examples of such polymers are made by Solvay Solexis and sold under the trade name Fomblin.

Also suitable are linear fluorinated polymers like those having the general formula:

HF₂C(OCF₂CF₂)ₓ(OCF₂)_{y}OCF₂H

Wherein x and y are as above defined.

Also suitable are linear fluorinated hydroxyl-substituted polymers like those having the general formula:

HOCH₂CF₂O(CF₂CF₂O)ₚ(CF₂O)_{q}CF₂CH₂OH

Wherein p and q are each independently 1 to 10,000.

Also suitable are organic polymers which are copolymers of acrylates, methacrylates or their simple C₁₋₄₀ straight or branched chain alkyl or perfluoroalkyl esters, which may be copolymerized with various organic groups such as repeating ethylene oxide (EO) or propylene oxide (PO) units, where the repeating units range from 0 to about 50,000. Examples of such polymers include acrylates/methoxy PEG-23 methacrylate/perfluorooctyl ethyl acrylate copolymer, acrylates/trifluoropropylmethacrylate/polytrimethyl siloxymethacrylate copolymer, behenyl methacrylate/perfluorooctylethyl methacrylate copolymer, C₆₋₁₄ perfluoroalkylethyl acrylate/HEMA copolymer, hexafluoropropylene/tetrafluoroethylene copolymer, stearyl methacrylate/perfluorooctylethyl methacrylate copolymer, trifluoroethyl methacrylate, and the like.

2. Fluorinated Silicone Polymers

Also suitable as the fluorinated polymer are various types of fluorinated silicones. Such silicones are generally linear, branched or crosslinked siloxanes comprised of M, D, T, and Q units.

The term “M” means a monofunctional siloxy unit, having the general formula:

(CH₃)₃SiO_{0.5}

with the “½” meaning that the silicon atom is bonded to one oxygen atom when the unit is polymerized with another D, T, or Q unit to form a siloxane polymer. Any one or more of the methyl groups on the monofunctional unit may be substituted with perfluoroalkyl groups. In the case where the monofunctional unit is substituted with one or more perfluoroalkyl groups or other groups the M′ (M prime) designation may be used. In the case where the M unit is M′, the general formula may be:

R₁R₂R₃SiO_{1/2}

Wherein each of R₁, R₂, and R₃ are independently methyl, C₁₋₁₀ straight or branched chain alkyl, PE, C₁₋₁₀ straight or branched chain perfluoroalkyl with the proviso that at least one of R₁, R₂, or R₃ must be other than methyl and wherein PE means —[O—(CR₂)ₐ]ₓ; wherein a is 2 to 6, and x is 1 to 5,000

The term “D unit” refers to a difunctional siloxy unit with the designation “2/2” meaning that the silicon atom is bonded to two oxygen atoms when the unit is polymerized with other units to form an organosiloxane polymer. The difunctional siloxy unit may have the general formula:

R₁R₂SiO_{2/2}

wherein R₁ and R₂ are each independently as set forth above. In the D unit the R substituents may be other than methyl, in which case D′ (D prime) may be used to denote the unit.

The letter “T” refers to a trifunctional siloxy unit, that is a siloxy unit that is bonded to three oxygen atoms when the unit is polymerized with any one or more of the other units to form an organosiloxane polymer. A T unit generally as the formula:

R₁SiO_{3/2}

wherein R₁ is as defined above. In the case where the T is substituted with substituents other than methyl, the T′ (T prime) designation is sometimes used.

The letter “Q” refers to a quadrifunctional siloxy unit, that is a siloxy unit that is bonded to four silicon atoms. A Q unit is of the formula:

SiO_{4/2}

with the 4/2 designation meaning that the silicon atom is bonded to four oxygen atoms when the unit is polymerized with one or more of the other units to form an organosiloxane polymer.

The fluorinated siloxane polymers that may be used in the compositions of the invention may contain any combination of M, D, T, or Q units, provided that one or more of the units contain fluorine substitutions.

Suitable organosiloxane polymers include those having the general formula:

R₁R₂R₃Si—{O—Si(CH₃)₂}ₘ—{O—Si(R)[(CH₂)ₓ(CF₂)_{y}—PE-CR₃]}ₙ—{O—SiRA}ₒ—O—Si(R₁R₂R₃)

wherein R₁, R₂, and R₃ are as defined above, m, n, and o are from 0 to 50,000 with the proviso that at least one of m, n, or o must be present; PE is —{O—[(CR₁)₂]ₐ}ₓ; wherein a is 2 to 6, and x is 1 to 50,000; R is methyl, perfluoro C₁₋₁₀ alkyl, x is 0 to 5,000, y is 0 to 5,000, z is 0 to 50,000; and A is —(CR₂)ₓ—C(O)—O—(CR₂)_{y}—(CF₂)_{z}—B or —(CH₂)ₓ—(CF₂)_{y}—B
wherein B is methyl or C₁₋₁₀ perfluoroalkyl.

Further examples of perfluorinated organosiloxane polymers include those having the CTFA names PEG-10 nonafluorohexyldimethicone copolymer, PEG-4, 8, or 10 trifluoroproyldimethicone copolymer, perfluorononyldimethicone, perfluoronony carboxyldecyl behenyl dimethicone, perfluorononyl carboxydecyl hexacosyl dimethicone, perfluorononylethyl carboxyldecyl lauryl/behenyl dimethicone, perfluorononylethyl carboxydecyl lauryl dimethicone, perfluorononylethyl carboxyldecyl PEG-8 dimethicone, perfluorononylethyl carboxydecyl PEG-10 dimethicone, perfluorononylethyldimethicone/methicone copolymer, perfluorononylethyl PEG-8 dimethicone, perfluorononylethyl PEG-8 dimethicone, perfluorononylethyl stearyldimethicone, trifluoro₁₋₄ alkyl dimethicone, trifluorocyclopentasiloxane, trifluorocyclotetrasiloxane, trifluoropropyldimethicone, trifluoropropyl PEG-10 dimethicone crosspolymer, trifluoropropyldimethicone/trifluoropropyldivinylmethicone crosspolymer, trifluoropropyl dimethicone/vinyl trifluoropropyl dimethicone/silsesquioxane copolymer, trifluoropropyl dimethiconol, trifluoropropyldimethyl/trimethylsiloxysilicate, C4-14 perfluoroalkylethoxy dimethicone trifluoropropylmethicone, C20-28 alkyl perfluorodecylethoxy dimethicone, dimethiconol fluoroalcohol dilinoleic acid and the like. Such silicones may be purchased from Siltech under the trade name Fluorosil; or from Shin-Etsu under the FPD designation, or from Dow Corning under the FS designation, or from Grant Industries under the Gransil trade name.

3. Fluorinated Silicone/Organic Polymers

Also suitable are various types of fluorinated copolymers of silicone and organic groups. Such polymers generally comprise one or more the M, D, T, or Q units mentioned above, copolymerized with one or more organic groups such as acrylic acid, methacrylic acid and their C₁₋₄₀ simple esters. The acrylic or methacrylic acid esters may exhibit perfluoro groups as well as other groups such as mercapto, and the like. Examples of such copolymers include butyl acrylate/C₆₋₁₄ perfluoroalkylethyl acrylate/mercaptopropyl dimethicone copolymer, isobutylmethacrylate/trifluoroethylmethacrylate/bis-hydroxypropyl dimethicone acrylate copolymer, dimethiconol fluoroalcohol dilinoleic acid, and the like.

### II. The Aromatic Component

The composition of the invention contains at least one aromatic component present in an amount sufficient to compatibilize the fluorinated component in the composition of the invention. Suggested ranges of aromatic component will depend on the amount of fluorinated component that is present, but will generally range from about 0.1 to 90%, preferably from about 0.5 to 85%, more preferably from about 1 to 80% by weight of the total composition. If desired, the fluorinated component may be dispersed in the aromatic component prior to incorporation of the components into the cosmetic composition of the invention. In that case the amount of fluorinated component that is desired for use in the composition of the invention is mixed with a portion, or all, of the amount of aromatic component that is desired for use in the composition to form a pre-blend that can be added to the composition when it is being formulated. For example from 10 to 90 parts of the fluorinated component may be combined from 10 to 90 parts of the aromatic component to form the pre-blend.

The aromatic component may be a compound, oligomer, or polymer, or combinations thereof. The term “aromatic” when used herein means homonuclear or heteronuclear aromatic compounds. The term “homonuclear” means that the aromatic ring contains only carbon atoms, e.g. the aromatic ring is “homonuclear”. The term “heteronuclear” means that the aromatic ring contains at least one hetero atom, that is a carbon atom that is bonded to another atom that is not carbon, such as nitrogen, sulfur, phosphorus, and so on.

In one preferred embodiment the aromatic component is polar, e.g. has a measurable dipole moment. A dipole moment may be measured by methods well known in the art.

A. Aromatic Compounds

The aromatic component may be an aromatic compound. The term “aromatic compound” means a compound containing at least one aromatic ring. The aromatic compound is present in an amount sufficient to solvate, or compatibilize the fluorinated component in the composition. Thus, in one embodiment of the invention the fluorinated component when used alone in the composition is not compatible with the other cosmetic ingredients used in the composition, without the aromatic component. The aromatic compound may have a molecular weight ranging from about 100 to 10,000. Preferably from about 100 to 600, more preferably from about 100 to 500 g/mole.

1. Aromatic Chemical Sunscreens

Examples of suitable aromatic compounds include compounds that are also FDA monograph approved UVA or UVB chemical sunscreens.

For example, dibenzoylmethane compounds are UVA sunscreens and have the general formula

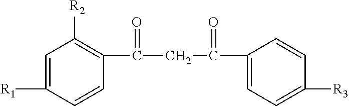

wherein R₁ is H, OR and NRR wherein each R is independently H, C₁₋₂₀ straight or branched chain alkyl; R₂ is H or OH; and R₃ is H, C₁₋₂₀ straight or branched chain alkyl are suitable as the aromatic compound used to compatibilize the fluorinated component. In one preferred embodiment, R₁ is OR where R is a C₁₋₂₀ straight or branched alkyl, preferably methyl; R₂ is H; and R₃ is a C₁₋₂₀ straight or branched chain alkyl, more preferably, butyl.

Preferred is 4-tert-butyl-4′-methoxydibenzoylmethane, also referred to as Avobenzone. Avobenzone has a molecular weight of about 310 g/mole and is commercial available from Givaudan-Roure under the trademark Parsol 1789, and Merck & Co. under the trade name Eusolex 9020. Other specific examples also include 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 2-methyl-5-tert-butyl-4′-methoxydibenzoylmethane, and so on.

Other suitable sunscreens include benzylidene camphor derivatives as set forth in U.S. Pat. No. 3,781,417, which is hereby incorporated by reference in its entirety. Such benzylidene camphor derivatives have the general formula:

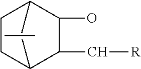

wherein R is p-tolyl or styryl, preferably styryl. Particularly preferred is 4-methylbenzylidene camphor, which is a lipid soluble UVB sunscreen compound sold under the tradename Eusolex 6300 by Merck.

Derivatives of cinnamic acid, including those having the general formula:

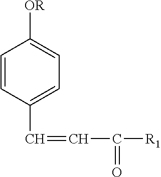

wherein R and R₁ are each independently a C₁₋₃₀ straight or branched chain alkyl. A particularly preferred compabilizer used in the compositions of the invention is ethylhexyl methoxycinnamate, also referred to as Octoxinate or octyl methoxycinnamate, having a molecular weight of about 290 g/mole. The compound may be purchased from Givaudan Corporation under the tradename Parsol MCX, or BASF under the tradename Uvinul MC 80. Mono-, di-, and triethanolamine derivatives of such methoxy cinnamates including diethanolamine methoxycinnamate, or Cinoxate, which is an aromatic ether derivative of the above compound may be used.

Another type of aromatic compound may include various benzophenone derivatives having the general formula:

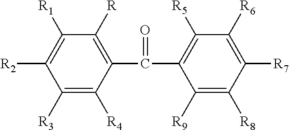

wherein R through R₉ are each independently H, OH, NaO₃S, SO₃H, SO₃Na, Cl, R″, OR″ where R″ is C₁₋₂₀ straight or branched chain alkyl Examples of such compounds include Benzophenone 1, 2, 3, 4, 5, 6, 7, 8, 9, 10, 11, and 12. Benzophenone 3 (also referred to as Oxybenzone) has a molecular weight of about 322 g/mole, Benzophenone 4 (also referred to as Sulisobenzone) has a molecular weight of about 304 g/mole, and Benzophenone 5 (Sulisobenzone Sodium) has a molecular weight of about 310 g/mole, and the like, may be suitable.

Also suitable are certain menthyl salicylate derivatives having the general formula:

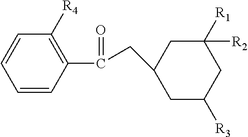

wherein R₁, R₂, R₃, and R₄ are each independently H, OH, NH₂, or C₁₋₂₀ straight or branched chain alkyl. Preferred is where R₁, R₂, and R₃ are methyl and R₄ is hydroxyl or NH₂. Specific examples of compounds are homomethyl salicylate (also known as Homosalate) having a molecular weight of about 262 g/mole, or menthyl anthranilate having a molecular weight of about 275 g/mole. Homosalate is available commercially from Merck under the tradename Eusolex HMS and menthyl anthranilate is commercially available from Haarmann & Reimer under the trade name Heliopan.

Also suitable are various amino benzoic acid derivatives including those having the general formula:

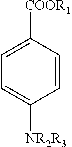

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Examples of such compounds include PABA (paraamino benzoic acid), ethyl hexyl dimethyl PABA (also known as Padimate O) which has a molecular weight of about 277 g/mole, ethyldihydroxypropyl PABA, and the like.

Salicylate derivatives may also be acceptable. Examples of such compounds include those having the general formula:

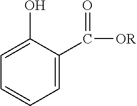

wherein R is a straight or branched chain alkyl, including derivatives of the above compound formed from mono-, di-, or triethanolamines, for example octyl salicylate, TEA-salicylate, DEA-salicylate, and the like. On preferred salicylate derivative is octylmethoxy cinnamate, which has a molecular weight of about 250 g/mole.

Also suitable are alpha-cyano-β,β-diphenyl acrylic acid esters, in particular those having the general formula:

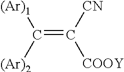

wherein each Ar is independently a homonuclear or heteronuclear aromatic ring, and Y is a straight or branched chain, saturated or unsaturated alkyl having from about 1 to 40 carbon atoms. Examples of such polymers are as set forth in U.S. Pat. No. 3,215,724, which is hereby incorporated by reference in its entirety. A particularly preferred compatibilizer is an alpha-cyano-beta,beta-diphenyl acrylic acid ester referred to as octocrylene, which is 2-ethylhexyl 2-cyano-3,3-diphenylacrylate having the structural formula:

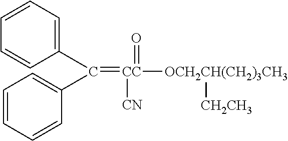

Octocrylene has the molecular weight of about 361 grams/mol, and may be purchased from BASF under the tradename Uvinul N-539.

Another suitable aromatic compound is drometrizole having the general structure:

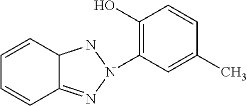

or drometrizole trisiloxane (also referred to as Mexoryl XL) having the structure:

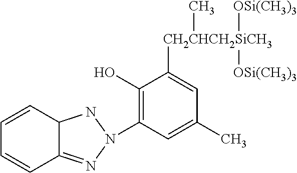

An aromatic compound referred to as Neo Heliopan, having the chemical name disodium phenyldibenzimidazole toluenesulfonate may also be suitable. This compound has the structure:

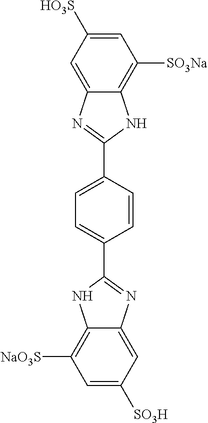

Also suitable is an aromatic compound referred to as Eusolex 232, having the chemical name diethyl butamidetriazine, and the structure:

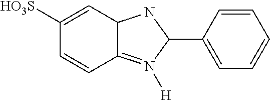

This compound may be purchased from EMD Chemicals, or from DSM Nutritional Products under the trade name Parsol HS, or from Symrise under the trade name Neo Heliopan HS.

2. Other Aromatic Compounds

A variety of other aromatic compounds may be used as the solubilizer or compatibilizer for the fluorinated component, including various benzene derivatives, including but not limited to those having the general formula:

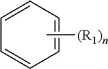

wherein n is 0 to 6, and each R₁ is independently hydrogen, chlorine, hydroxyl, C₁₋₄₀ straight or branched chain saturated or unsaturated alkyl, C₁₋₄₀ alkyloxy C₁₋₄₀ alkanol, C₁₋₄₀ alkyloxy C₁₋₄₀ alkanol, C₁₋₄₀ alkyloxycarbonyl C₁₋₄₀ alkyl, C₁₋₄₀ alkanol, C₁₋₄₀ alkoxy, C₁₋₄₀ alkyloxycarbonylpyrimidine, carboxyoxyaryl carboxyoxy C₁₋₄₀ alkylary, carboxyoxy C₁₋₄₀ alkyl, carboxyoxy C₁₋₄₀ alkylaryl, pyrrolidone, and the like.

Examples of such compounds include, but are not limited to, benzyl acetate, benzyl alcohol, benzyl benzoate, benzylbenzyloxybenzoate, benzyl glycol, benzyl hemiformal, benzylheptanol, benzyl laurate, benzyl laurate/myristate/palmitate, benzyl nicotinate, benzyl PCA, dichlorobenzyl alcohol, dimethoxybenzyl alcohol, hydroxymethoxybenzyl pelargonamide, PPG-benzyl ether myristate, and so on.

B. Aromatic Polymers

1. Aromatic Organic Polymers

Other aromatic polymers include polymers of acrylic acid, methacrylic acid or their simple aromatic or aliphatic C₁₋₄₀ straight or branched chain saturated or unsaturated esters; either alone or copolymerized with organic groups such as alkylene glycol, amide, and the like. Such polymers have at least one homo- or heteronuclear aromatic group. Examples of such polymers include acrylates/octylacrylamide/diphenyl amodimethicone copolymer, and the like.

2. Aromatic Silicones

Also suitable as the aromatic polymer are one or more silicones. Examples of such silicones include silicones substituted with phenyl groups such as diphenyl dimethicone, phenyl trimethicone, trimethylsiloxyphenyldimethicone, phenyl dimethicone and so on.

III. Other Ingredients

The composition of the invention may contain a variety of other ingredients. The cosmetic compositions may be in the liquid, solid, or semi-solid form. They may aqueous based or anhydrous. If the former, they may be found in the form of an aqueous solution, suspension or dispersion, emulsion, or gel. If the form of an emulsion, they may be water-in-oil or oil-in-water emulsions.

A. Oils

The composition may contain one or more oils. The term “oil” when used herein refers to a material that is pourable at room temperature (25° C.). Suitable oils may be volatile or nonvolatile. The term “volatile” when used herein means that the oil has a vapor pressure of greater than about 2 mm. of mercury at 20° C. The term “nonvolatile” means that the oil has a vapor pressure of less than 2 mm. of mercury at 20° C. If present, the oils may range from about 0.1 to 90%, preferably from about 0.5 to 85%, more preferably from about 1 to 80% by weight of the total composition.

The composition of the invention is in the emulsion form and comprises an oil phase. Suitable oils include silicones, esters, vegetable oils, synthetic oils, including but not limited to those set forth herein. The oils may be volatile or nonvolatile, and are in the form of a pourable liquid at room temperature. The term “volatile” means that the oil has a measurable vapor pressure, or a vapor pressure of at least about 2 mm. of mercury at 20° C. The term “nonvolatile” means that the oil has a vapor pressure of less than about 2 mm. of mercury at 20° C.

1. Volatile Oils

(a). Volatile Silicones

Suitable volatile oils generally have a viscosity ranging from about 0.5 to 5 centistokes 25° C. and include linear silicones, cyclic silicones, paraffinic hydrocarbons, or mixtures thereof.

Cyclic silicones are of the general formula:

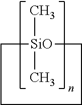

where n=3-6.

Linear or branched volatile silicones in accordance with the invention have the general formulas:

(CH₃)₃Si—O—[Si(CH₃)₂—O]ₙ—Si(CH₃)₃

where n=0, 1, 2, 3, 4, or 5, preferably 0, 1, 2, 3, or 4; or

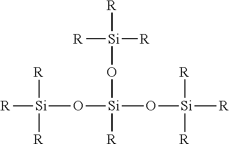

wherein R is C₁₋₄ alkyl, preferably methyl.

Linear, branched, and cyclic volatile silicones are available from various commercial sources including Dow Corning Corporation, GE Silicones, and Shin-Etsu. The Dow Corning volatile silicones are sold under the tradenames Dow Corning 244, 245, 344, and 200 fluids. These fluids comprise octamethylcyclotetrasiloxane, decamethylcyclopentasiloxane, dodecamethylcyclohexasiloxane and the like. Suitable linear volatile silicones include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof. Suitable branched volatile silicones include methyl trimethicone, ethyl trimethicone, propyl trimethicone, butyl trimethicone and the like. Methyl trimethicone may be purchased from Shin-Etsu Silicones and has the trade name TMF 1.5, having the viscosity of 1.5 centistokes at 25° C.

(b). Volatile Paraffinic Hydrocarbons

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins such as isododecane. Such hydrocarbons are taught in U.S. Pat. Nos. 3,439,088 and 3,818,105, both of which are hereby incorporated by reference.

Preferred volatile paraffinic hydrocarbons have a molecular weight of 75 to 225, preferably 160 to 195 and a boiling point range of about 60 to 260° C., and a viscosity of less than about 10 cst. at 25° C. Such paraffinic hydrocarbons are available from EXXON under the ISOPARS trademark, and from the Permethyl Corporation. Suitable C₁₂ isoparaffins such as isododecane are manufactured by Permethyl Corporation under the tradename Permethyl 99A. Various C₁₆ isoparaffins commercially available, such as isohexadecane (having the tradename Permethyl R), are also suitable.

2. Non-Volatile Oils

A variety of nonvolatile oils are also suitable for use in the cosmetic compositions of the invention. The nonvolatile oils generally have a viscosity of greater than about 5 to 10 centistokes at 25° C., and may range in viscosity up to about 1,000,000 centipoise at 25° C. Examples of nonvolatile oils include, but are not limited to:

(a). Esters

Suitable esters are mono-, di-, and triesters. The composition may comprise one or more esters selected from the group, or mixtures thereof.

(i). Monoesters

Monoesters are defined as esters formed by the reaction of a monocarboxylic acid having the formula R—COOH, wherein R is a straight or branched chain saturated or unsaturated alkyl having 2 to 45 carbon atoms; and an alcohol having the formula R—OH wherein R is a straight or branched chain saturated or unsaturated alkyl having 2-30 carbon atoms. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, steary lactate, stearyl octanoate, stearyl stearate, isononyl isononanoate, and so on.

(ii). Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

(iii). Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described on pages 1670-1676 of the C.T.F.A. Cosmetic Ingredient Dictionary and Handbook, Eighth Edition, 2000, which is hereby incorporated by reference in its entirety.

(b). Hydrocarbon Oils

It may be desirable to incorporate one or more nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins such as hydrogenated or non-hydrogenated polyisobutene, polyisobutene, polydecene; mineral oil, pentahydrosqualene, squalene, squalane, and mixtures thereof. In one preferred embodiment such hydrocarbons have a molecular weight ranging from about 300 to 1000 Daltons.

(c). Glyceryl Esters of Fatty Acids

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups, glyceryl stearate, diglyceryl diiosostearate, polyglyceryl-2 isostearate, polyglyceryl-2 triisostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(d). Nonvolatile Silicones

Nonvolatile silicone oils, both water soluble and water insoluble, are also suitable for use in the composition. Such silicones preferably have a viscosity ranging from about 10 to 800,000 cst, preferably 20 to 200,000 cst at 25° C. Suitable water insoluble silicones include amine functional silicones such as amodimethicone; dimethicone, dimethicone substituted with C₂₋₃₀ alkyl groups such cetyl or lauryl dimethicone.

Nonvolatile silicones may have the following general formula:

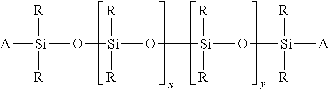

wherein R and R¹ are each independently C₁₋₃₀ straight or branched chain, saturated or unsaturated alkyl, or aryl, trialkylsiloxy, and x and y are each independently 0-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, or trimethylsiloxy, more preferably a C₁₋₂₂ alkyl, or trimethylsiloxy, most preferably methyl or trimethylsiloxy, and resulting silicone is dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Cetyl dimethicone, also referred to as a liquid silicone wax, may be purchased from Dow Corning as Fluid 2502, or from DeGussa Care & Surface Specialties under the tradenames Abil Wax 9801, or 9814.

B. Structuring Agents

A variety of structuring agents may be present. The term “structuring agent” means an ingredient or combination of ingredients, soluble or dispersible in either the oil phase, the water phase (if present), or both, which will increase the viscosity, or structure, the composition. In the case where the composition is anhydrous, the structuring agent, if present, will generally be an oil phase structuring agent. In the case where the composition is in an aqueous form, the structuring agent may be found in the water phase. If the composition is in the form of an emulsion, the structuring agent may be found in either one, or both phases.

The structuring agent itself may be present in the liquid, semi-solid, or solid form. Suggested ranges of structuring agent, present, are from about 0.01 to 70%, preferably from about 0.05 to 50%, more preferably from about 0.1-35% by weight of the total composition. Suitable structuring agents include those that are silicone based or organic based. They may be polymers or non-polymers, synthetic, natural, or a combinations thereof.

1. Silicone Structuring Agents

Silicone based structuring agents are suitable, such as silicone elastomers, silicone gums, silicone waxes, linear silicones having a degree of polymerization that provides the silicone with a degree of viscosity such that when incorporated into the cosmetic composition it is capable of increasing the viscosity of the composition. Examples of silicone structuring agents include, but are not limited to:

(a). Silicone Elastomers

Silicone elastomers suitable for use in the compositions of the invention include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxanes, and dimethylvinylsiloxy-terminated dimethylsiloxane-methyl(3,3-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or nonvolatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105 and Dow Corning's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety.

(b). Silicone Gums

Also suitable for use as a structuring agent are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million cst. All ranges mentioned herein include all subranges and integers therebetween, e.g. 550,000; 925,000; 3.5 million.

The silicone gums that are used in the compositions include, but are not limited to, those of the general formula:

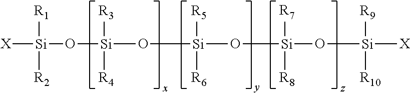

wherein, R₁ to R₉ are each independently an alkyl having 1 to 30 carbon atoms, and X is OH or a C₁₋₃₀ alkyl, or vinyl; and wherein x, y, or z may be zero with the proviso that no more than two of x, y, or z are zero at any one time, and further that x, y, and z are such that the silicone gum has a viscosity of at least about 500,000 cst, ranging up to about 100 million cst at 25° C. Preferred is where R is methyl or OH.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the CTFA name dimethicone.

(c). Silicone Waxes

Another type of structuring agent includes silicone waxes that are typically referred to as alkyl silicone waxes which are semi-solids or solids at room temperature. The term “alkyl silicone wax” means a polydimethylsiloxane having a substituted long chain alkyl (such as C16 to 30) that confers a semi-solid or solid property to the siloxane. Examples of such silicone waxes include stearyl dimethicone, which may be purchased from DeGussa Care & Surface Specialties under the tradename Abil Wax 9800 or from Dow Corning under the tradename 2503. Another example is bis-stearyl dimethicone, which may be purchased from Gransil Industries under the tradename Gransil A-18, or behenyl dimethicone, behenoxy dimethicone.

2. Polyamides or Silicone Polyamides

Also suitable as oil phase structuring agents are various types of polymeric compounds such as polyamides or silicone polyamides.

The term silicone polyamide means a polymer comprised of silicone monomers and monomers containing amide groups as further described herein. The silicone polyamide preferably comprises moieties of the general formula:

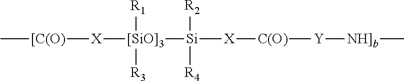

wherein X is a linear or branched alkylene having from about 1-30 carbon atoms;
R₁, R₂, R₃, and R₄ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

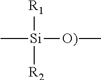

### and Y is:

(a) a linear or branched alkylene having from about 1-40 carbon atoms which may be substituted with (i) one or more amide groups having the general formula R₁CONR₁, or (ii) C₅₋₆ cyclic ring, (ii) hydroxy, or (iii) C₃₋₈ cycloalkane, or (iv) C₁₋₂₀ alkyl which may be substituted with one or more hydroxy groups, or (v) C₁₋₁₀ alkyl amines; or

(b) TR₅R₆R₇

wherein R₅, R₆, and R₇, are each independently a C₁₋₁₀ linear or branched alkylenes, and T is CR₈ wherein R₈ is hydrogen, a trivalent atom N, P, or Al, or a C₁₋₃₀ straight or branched chain alkyl which may be substituted with one or more hydroxyl or halogen groups; halogen, hydroxyl, or alkoxy groups; or a siloxane chain having the general formula:

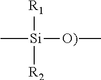

Preferred is where R₁, R₂, R₃, and R₄ are C₁₋₁₀, preferably methyl; and X and Y is a linear or branched alkylene. Preferred are silicone polyamides having the general formula:

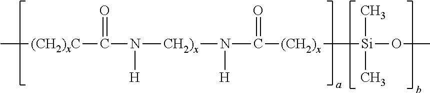

wherein a and b are each independently sufficient to provide a silicone polyamide polymer having a melting point ranging from about 60 to 120° C., and a molecular weight ranging from about 40,000 to 500,000 Daltons. One type of silicone polyamide that may be used in the compositions of the invention may be purchased from Dow Corning Corporation under the tradename Dow Corning 2-8178 gellant which has the CTFA name nylon-611/dimethicone copolymer which is sold in a composition containing PPG-3 myristyl ether.

Also suitable are polyamides such as those purchased from Arizona Chemical under the trade names Uniclear and Sylvaclear. Such polyamides may be ester terminated or amide terminated. Examples of ester terminated polyamides include, but are not limited to those having the general formula:

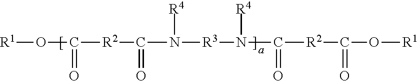

wherein n denotes a number of amide units such that the number of ester groups ranges from about 10% to 50% of the total number of ester and amide groups; each R₁ is independently an alkyl or alkenyl group containing at least 4 carbon atoms; each R₂ is independently a C₄₋₄₂ hydrocarbon group, with the proviso that at least 50% of the R₂ groups are a C30-42 hydrocarbon; each R₃ is independently an organic group containing at least 2 carbon atoms, hydrogen atoms and optionally one or more oxygen or nitrogen atoms; and each R₄ is independently a hydrogen atom, a C₁₋₁₀ alkyl group or a direct bond to R₃ or to another R₄, such that the nitrogen atom to which R₃ and R₄ are both attached forms part of a heterocyclic structure defined by R₄—N—R₃, with at least 50% of the groups R₄ representing a hydrogen atom.

General examples of ester and amide terminated polyamides that may be used as oil phase gelling agents include those sold by Arizona Chemical under the tradenames Sylvaclear A200V or A2614V, both having the CTFA name ethylenediamine/hydrogenated dimer dilinoleate copolymer/bis-di-C₁₄₋₁₈ alkyl amide; Sylvaclear AF1900V; Sylvaclear C75V having the CTFA name bis-stearyl ethylenediamine/neopentyl glycol/stearyl hydrogenated dimer dilinoleate copolymer; Sylvaclear PA1200V having the CTFA name Polyamide-3; Sylvaclear PE400V; Sylvaclear WF1500V; or Uniclear, such as Uniclear 100VG having the INCI name ethylenediamine/stearyl dimer dilinoleate copolymer; or ethylenediamine/stearyl dimer ditallate copolymer. Other examples of suitable polyamides include those sold by Henkel under the Versamid trademark (such as Versamid 930, 744, 1655), or by Olin Mathieson Chemical Corp. under the brand name Onamid S or Onamid C.

3. Natural or Synthetic Organic Waxes

Also suitable as the structuring agent may be one or more natural or synthetic waxes such as animal, vegetable, or mineral waxes. Preferably such waxes will have a higher melting point such as from about 60 to 150° C., more preferably from about 65 to 100° C. Examples of such waxes include waxes made by Fischer-Tropsch synthesis, such as polyethylene or synthetic wax; or various vegetable waxes such as candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.

4. Montmorillonite Minerals

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like.

5. Silicas and Silicates

Another type of structuring agent that may be used in the compositions are silicas, silicates, silica silylate, and alkali metal or alkaline earth metal derivatives thereof. These silicas and silicates are generally found in the particulate form and include silica, silica silylate, magnesium aluminum silicate, and the like.

6. Polysaccharides

A variety of polysaccharides may be suitable structuring or thickening agents, particularly if the composition of the invention is found in the aqueous form. Examples of such polysaccharides include naturally derived materials such as agar, agarose, alicaligenes polysaccharides, algin, alginic acid, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

7. Acrylate Polymers

Acrylic polymeric thickeners are suitable, particularly if the composition is aqueous based. For example, acrylic polymeric thickeners comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof are suitable. In one embodiment the A monomer comprises one or more of acrylic acid or methacrylic acid, and the B monomer is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. The acrylic copolymer may be supplied in an aqueous solution having a solids content ranging from about 10-60%, preferably 20-50%, more preferably 25-45% by weight of the polymer, with the remainder water. The composition of the acrylic copolymer may contain from about 0.1-99 parts of the A monomer, and about 0.1-99 parts of the B monomer. Acrylic polymer solutions include those sold by Seppic, Inc., under the tradename Capigel.

Also suitable are acrylic polymeric thickeners that are copolymer of A, B, and C monomers wherein A and B are as defined above, and C has the general formula:

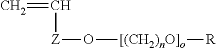

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid or mixtures thereof, and where the allyl ether unit containing a fatty chain corresponds to the monomer of formula

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C18) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl(meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 alkyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C₁₀₋₃₀ alkyl acrylate crosspolymer which is a copolymer of C10-30 alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames.

Particularly suitable as the aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

8. High Molecular Weight PEG or Polyglycerins

Also suitable as structuring agents are various polyethylene glycols (PEG) derivatives where the degree of polymerization ranges from 10,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CFTA names polyglycerin-20, polyglycerin-40, and the like.

The compositions of the invention may contain particulate materials in the form of pigments, inert particulates, or mixtures thereof. If present, suggested ranges are from about 0.1-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

C. Particulates

The composition may comprise particulates in the form of pigments, powders, or fillers. If present, such particulates may range from about 0.1 to 99%, preferably from about 0.5 to 95%, more preferably from about 1 to 85% by weight of the total composition.

1. Powders

The particulate matter may be colored or non-colored (for example white or colorless) non-pigmented powders. Suitable non-pigmentated powders include bismuth oxychloride, titanated mica, fumed silica, spherical silica, polymethylmethacrylate, micronized teflon, boron nitride, acrylate copolymers, PMMA, barium sulfate, aluminum hydroxide, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. Such powders may be surface treated with lecithin, amino acids, mineral oil, silicone, or various other agents either alone or in combination, which coat the powder surface and render the particles more lipophilic in nature.

2. Pigments

The particulate materials may comprise various organic or inorganic pigments or both. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

Suitable inorganic pigments include the iron oxides, such as red, yellow, black, and the like.

The composition may contain a mixture of pigments and powders, and in such case ranges of from about 0.1 to 40% pigment and from about 0.1 to 60% powder may be suitable.

D. Botanical Extracts

It may be desirable to include one or more botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including soybean extract, yeast ferment extract, padica pavonica extract, thermus thermophilis ferment extract, camelina sativa seed oil, boswellia serrata extract, olive extract, aribodopsis thaliana extract, acacia dealbata extract, acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2.

E. Film Formers

It may be desirable to include one or more film formers in the composition. If present suggested ranges are from about 0.1 to 60%, preferably from about 0.5 to 50%, more preferably from about 1 to 45% by weight of the total composition. The term “film former” means an ingredient that will form some type of film on the keratinous surface to which it is applied. The film formers may be natural or synthetic, and in the monomeric or polymeric form. Suitable film forming polymers include copolymers of vinyl pyrrolidone and paraffinic hydrocarbons, e.g. VP/eicosene, PVP/triacontene, butylated PVP, PVP/hexadecane copolymer, VA/butyl maleate/isobornyl acrylate copolymer, VP/acrylates/lauryl methacrylate copolymer, acrylates/C1-2 succinates/hydroxyacrylates copolymer, acrylates/C 12-22 alkyl methacrylate copolymer, styrene/VP copolymer, isobutylene/ethylmaleimide/hydroxyethylmaleimide copolymer, VP/vinyl caprolactam/DMAPA acrylates copolymer, Polyimide-1, isobutylene/ethylmaleimide/hydroxyethylmaleimide copolymer, VP/dimethylaminoethylmethacrylate copolymer, vinyl caprolactam/VP/dimethylaminoethyl methacrylate copolymer, PVM/MA copolymer, butyl ester of PVM/MA copolymer, ethyl ester of PVM/MA copolymer, PVP, PVP/VA copolymer, and the like.

Also suitable are silicone based film formers such as silicone resins. Examples of such resins include trimethylsiloxysilicate, which may be purchased from Dow Corning Corporation under the tradename 749 Fluid, which is an approximately equal portion of volatile cyclic silicone and trimethylsiloxysilcate; or from GE Silicones under the trade name SR-1000. Another type of silicone resin is polymethylsilsesquioxane, commonly referred to as an MT resin, meaning that it largely contains monofunctional and trifunctional units. One example of such resin can be purchased from Wacker-Chemie under the tradename MK resin.

Also suitable are certain film formers that are prepared by esterifying polyhydric alcohols, in particular those known as sugars. For example, aliphatic carboxylic acid esters having from about 1 to 22 carbon atoms, or aromatic carboxylic acid esters of sucrose, fructose, glucose, and the like are suitable film formers. One such example is sucrose acetate isobutyrate, which is a mixed ester of sucrose and acetic and isobutyric acids. Another example is sucrose acetate/stearate which is a mixed ester of sucrose and acetic and stearic acids.

F. Surfactants

It may be desirable to include one or more surfactants in the composition. If in the anhydrous form the surfactant may aid in the suspension or dispersion of pigments or particulates in the formula. If in the emulsion form, the surfactant may facilitate formation of a stable emulsion. Suitable surfactants are preferably nonionic in character and may comprise silicone surfactants, organic nonionic surfactants or combinations thereof. If present, suggested ranges are from 0.01 to 40%, preferably from about 0.1 to 35%, more preferably from about 0.5 to 25% by weight of the total composition.

Suitable silicone surfactants are linear or crosslinked siloxanes that contain at least one hydrophilic moiety. Examples of suitable silicone surfactants include those generically referred to as dimethicone copolyol, lauryl dimethicone copolyol, cetyl dimethicone copolyol, PEG2 to PEG300 dimethicones with the number after the PEG indicating the number of repeating ethylene glycol units, polyglyceryl 3 to 200 dimethicones with the number after the “polyglyceryl” referring to the number of repeating glyceryl moieties, e.g. polyglyceryl-3 dimethicone. Also suitable are crosslinked organosiloxane surfactants such as dimethicone/PEG-10 crosspolymer, dimethicone PEG-10/15 crosspolymer, diemthicone PEG-15 crosspolymer, dimethicone/polyglycerin-3 crosspolymer,

Suitable nonionic organic surfactants include polyethylene glycol ethers of fatty alcohols such as stearyl, cetyl, isostearyl, lauryl, myristyl, behenyl, alcohols and the like. For example, suitable surfactants include steareth 2-200, ceteth 2-200, ceteareth 2-200, ceteth 2-200, deceth 2-200, beheneth 2-200, laneth 2-200, laureth 2-200, myreth 2-200, and the like.

Also suitable are esters of polyethylene glycol ethers of fatty C₆₋₃₀ alcohols, for example, steareth-4 stearate, beheneth-10 behenate, steareth-10 stearate, and the like.

Also suitable as nonionic organic surfactants are mono and diesters of fatty acid such as oleic acid and hexitol anhydrides derived from sorbitol; or the polysorbates such as Polysorbates 20 to 85, trideceth 2 to 200, and so on.

G. Humectants

It may also be desirable to include one or more humectants in the composition. If present, suggested ranges are from about 0.1 to 40%, preferably from about 0.5 to 35%, more preferably from about 1 to 30% by weight of the total composition. Examples of suitable humectants include glycols, sugars, and the like. Suitable glycols include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol.

The composition may also contain other ingredients such as preservatives, botanicals, antioxidants, vitamins, and the like.

The composition may be in the form of solid sticks, cakes, creams, liquids, pastes, and the like.

The invention will be further described in connection with the following examples which are set forth for the purposes of illustration only.

### Example 1

A lipstick composition was made as follows:

| Ingredient | w/w % |
|---|---|
| Titanium dioxide | 1.62 |
| D&C Red No. 7 | 1.08 |
| FD&C Blue No. 1 Aluminum Lake | 0.03 |
| FD&C Yellow No. 5 Aluminum Lake | 1.56 |
| Iron oxides | 3.77 |
| Barium sulfate | 0.0001 |
| Aluminum hydroxide | 0.0001 |
| Hydrogenated polyisobutene | 8.94 |
| Polyglyceryl-2 triisostearate | 8.00 |
| VP/eicosene copolymer | 1.00 |
| Mica/titanium dioxide/iron oxides | 3.06 |
| Mica/titanium dioxide | 0.88 |
| Polyethylene | 10.00 |
| Dipalmitoyl hydroxyproline | 0.50 |
| Soybean extract/ceramide III | 1.00 |
| Tocopheryl linoleate | 0.50 |
| Nylon-12 | 3.00 |
| N-octyl perfluorononamide | 10.00 |
| Ethylhexyl methoxycinnamate | 5.00 |
| Octocrylene | 5.00 |
| Sucrose acetate isobutyrate | 1.00 |
| Isododecane | 25.00 |
| Methyl trimethicone | 6.06 |
| Isododecane/bis vinyl dimethicone/dimethicone copolymer | QS |

The composition was prepared by grinding the pigments in a portion of the nonvolatile oil. The fluorinated component was dispersed in the aromatic components. The waxes, and dispersed fluorinated component oils were heated and mixed well. The pigment grind was added to the molten composition and the volatile solvents added last. The molten composition was poured into molds and allowed to cool to form lipstick bullets. When applied to the lips the composition provided a shiny, transfer resistant finish.

### Example 2

An emulsion foundation makeup composition was prepared as follows:

| Ingredient | w/w % |
|---|---|
| Lecithin | 0.50 |
| Cetyl PEG/PPG-10/1 | 0.50 |
| dimethicone/polyglyceryl-4 isostearate/hexyl laurate | |
| HDI/trimethylol hexyllactone crosspolymer/silica | 8.00 |
| Butylene glycol | 4.00 |
| Silica | 0.90 |
| Water | QS |
| Disodium EDTA | 0.15 |
| Phenyl trimethicone/dimethicone/octyldodecyl stearoyl stearate | 10.50 |
| Glycerin | 4.00 |
| Dimethicone copolyol | 3.10 |
| Cyclomethicone/dimethicone/polysilicone-11/nylon-12/talc/silica/ | 1.25 |
| zinc oxide | |
| Phenoxyethanol/chlorophenesin/glycerin/sorbic acid | 1.50 |
| Trifluoromethyl Cl-4 alkyl dimethicone/cyclomethicone/propylene | 10.00 |
| carbonate/quaternium hectorite | |
| Laureth-7 | 0.10 |
| Ethylhexylglycerin | 0.50 |
| Mica/silica/dimethicone | 2.50 |
| Cyclopentasiloxane | 10.39 |
| Sodium chloride | 1.00 |
| Sodium dehydroacetate | 0.20 |
| Cyclomethicone/dimethicone/phenyl methicone | 4.00 |
| Magnesium aluminum silicate | 0.05 |
| Tocopheryl acetate | 0.10 |
| Xanthan gum | 0.10 |
| Iron oxides/C9-15 fluoroalcohol phosphates | 0.525 |
| Mica/silica | 0.10 |
| Titanium dioxide | 0.10 |

The water, oil and pigment phases were separately prepared by low shear mixing. The phases were combined with high shear blending to form a foundation makeup composition.

### Example 3

Lipstick compositions were prepared as follows:

| Ingredients | A | B | C |
|---|---|---|---|
| Titanium dioxide | 1.62 | 1.62 | 3.43 |
| D&C Red No. 7 Calcium Lake | 1.08 | 1.08 | 0.88 |
| FD&C Blue No. 1 Aluminum Lake | 0.03 | 0.03 | 0.12 |
| FD&C Yellow No. 5 Aluminum Lake | 1.56 | 1.56 | — |
| Iron oxides | 3.77 | 3.77 | 2.24 |
| Black iron oxides | — | — | 0.45 |
| Iron oxides/silica | — | — | 3.43 |
| Barium sulfate | 0.0001 | 0.0001 | — |
| Aluminum hydroxide | 0.0001 | 0.0001 | — |
| Hydrogenated polyisobutene | 8.94 | 8.94 | 14.00 |
| Polyglyceryl-2 triisostearate | 8.00 | 8.00 | 10.00 |
| VP/eicosene copolymer | 1.00 | 1.00 | — |
| Mica/titanium dioxide/iron oxides | 3.00 | 3.06 | 5.47 |
| Mica/titanium dioxide/iron oxides/ | — | — | 1.33 |
| carmine | | | |
| Mica/titanium dioxide | 0.88 | 0.88 | 2.61 |
| Mica/silica/dimethicone | — | — | 1.00 |
| Polyethylene | 10.00 | 10.00 | 10.00 |
| Dipentaerythrityl tripolyhydroxystearate | — | — | 2.00 |
| Dipalmitoyl hydroxyproline | 0.50 | 0.50 | — |
| Soybean extract/ceramide III | 1.00 | 1.00 | — |
| Tocopheryl linoleate/oleate | 0.50 | 0.50 | 0.80 |
| Nylon-12 | 3.00 | 3.00 | — |
| N-octylperfluorononamide | 10.00 | 10.00 | 5.00 |
| Ethylhexyl methoxycinnamate | 5.00 | 5.00 | — |
| Octocrylene | 2.00 | 5.00 | — |
| Sucrose acetate isobutyrate | 1.00 | 1.00 | — |
| Isododecane | QS | QS | QS |
| Methyl trimethicone | 6.10 | 6.10 | — |
| Cetyl PEG/PPG-10/1 | — | — | 2.00 |
| dimethcione/polyglyceryl-4 isostearate/ | | | |
| hexyl laurate | | | |
| Isododecane/bis-vinyl | 3.50 | 3.00 | — |
| dimethicone/dimethicone copolymer | | | |
| Phenylpropyldimethylsiloxysilicate | 0.50 | — | — |
| Methyl perfluorobutyl ether | — | 10.00 | — |
| Wheat bran extract/olive extract | — | — | 0.20 |

Compositions A and B were prepared by first dispersing the fluorinated component into the aromatic component. The pigments and particulates were ground in a portion of the oils. The dispersed fluorinated component, oils, waxes, and other ingredients were combined with heat and mixed with the pigment grind. The compositions were poured into lipstick molds and allowed to cool to room temperature. Composition C was prepared by grinding the pigment in a portion of the oils. The fluorinated component was combined with the other oils and waxes and the pigment grind, with heat, and mixed well. The molten composition was poured into molds and allowed to cool. Compositions A and B provided homogeneous transfer resistant lipsticks. In Composition C, the fluorinated ingredient was not compatible with the composition and phase separated upon application.

### Example 4

Emulsions of the fluorinated component in water were prepared as follows:

| Ingredient | w/w % | |
|---|---|---|
| CH₃—(CF₂)₇—CONH—CH₂— PEG₅₅₀ CH₃ | 3.8 | 41.7 |
| Ethyl perfluorobutyl ether | 76.9 | 16.7 |
| Water | QS 100 | QS100 |

The compositions were prepared by combining the ingredients and mixing well. Both formulas separated immediately and were internally incompatible.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

What is claimed is:

1. A cosmetic composition comprising at least one perfluorinated component and at least one aromatic component, wherein the aromatic component is present in an amount sufficient to compatibilize the fluorinated component in the cosmetic composition.

2. The composition of claim 1 wherein the perfluorinated component is a cosmetically incompatible perfluorinated compound.

3. The composition of claim 1 wherein the perfluorinated component is a compound.

4. The composition of claim 3 wherein the perfluorinated compound comprises a radical of the general formula (Radical I):
CXYZ—(CXY)ₐ—
wherein “a” ranges from 1 to 50,000, and each of X, Y, and Z are independently hydrogen, fluorine, C₁₋₁₀ alkoxy, C₁₋₁₀ alkyl, hydroxyl, halogens other than fluorine, C₁₋₁₀ alkoxy C₁₋₁₀ alkyl, and the like, with the proviso that Radical I contains at least one substituted fluorine atom.

5. The composition of claim 4 wherein the perfluorinated compound comprises a radical of the formula (Radical II):
C(O)—NR—; —C(O)—; —O—; —O—C(O)—; —C(O)—O—; or OH
Wherein R is hydrogen or a C₁₋₁₀ straight or branched chain alkyl.

6. The composition of claim 5 wherein the perfluorinated compound further comprises a radical of the formula (Radical III):
—(CMN)a-CLMN
wherein each L, M or N is independently hydrogen, hydroxyl, alkoxy, alkylalkoxyalkyl, alkylalkoxy, alkoxyalkyl, wherein the alkyl or alkoxy is a C₁₋₁₀ straight or branched chain; or alkylene glycol having the general formula:
—[O—(CH₂)ₓ]_{y}—
wherein x is 2, 3, 4, 5, 6, 7, 8, 9, or 10; preferably 2, 3, 4, or 5, more preferably 2, 3, or 4; and y ranges from 2 to 50,000, preferably from 2 to 400, more preferably from about 2 to 350.

7. The composition of claim 1 wherein the perfluorinated component is a perfluorinated amide.

8. The composition of claim 7 wherein the perfluorinated amide comprises at least one perfluoroalkyl group and at least one alkyl group wherein both groups are bonded to the at least one amide group.

9. The composition of claim 8 wherein the at least one perfluoroalkyl group is a C₁₋₃₀ straight or branched chain, saturated or unsaturated perfluoroalkyl group wherein one or more of the hydrogen atoms are substituted with fluorine atoms.

10. The composition of claim 9 wherein the perfluoroalkyl group all of the hydrogen atoms in the alkyl chain are substituted with fluorine atoms.

11. The composition of claim 1 wherein the perfluorinated component is N-octylperfluorononamide.

12. The composition of claim 1 wherein the aromatic component is an aromatic compound.

13. The composition of claim 12 wherein the aromatic compound is a chemical sunscreen.

14. The composition of claim 13 wherein the aromatic component is Octocrylene, ethylhexylmethoxycinnamate, or mixtures thereof.

15. A transfer resistant color cosmetic composition comprising at least one perfluorinated component, at least one aromatic component, at least one structuring agent, at least one oil, and particulates.

16. The composition of claim 15 wherein the perfluorinated component is a perfluorinated compound and the aromatic component is an aromatic compound.

17. The composition of claim 16 wherein the perfluorinated compound is a perfluorinated amide and the aromatic compound is a chemical sunscreen.

18. The composition of claim 15 wherein the perfluorinated component is N-octylperfluorononamide, the aromatic component is Octocrylene, ethylhexyl methoxycinnamate, or mixtures thereof.

19. A method for compatibilizing a fluorinated component with the ingredients in a cosmetic composition comprising formulating the composition with compatibilizing effective amount of an aromatic component.

20. A cosmetic composition comprising at least one perfluorinated amide.
